(19) United States
(12) Patent Application Publication
(10) Pub. No.: US 2026/0109921 A1
(43) Pub. Date: Apr. 23, 2026

(54) PREDICTION METHOD AND PREDICTION DEVICE

(71) Applicant: Hitachi, Ltd., Chiyoda-ku, Tokyo (JP)

(72) Inventors: Shiori NAKAZAWA, Tokyo (JP); Midori KATO, Tokyo (JP); Hikaru SAITO, Tokyo (JP); Hiroko HANZAWA, Tokyo (JP); Shizu TAKEDA, Tokyo (JP)

(21) Appl. No.: 19/118,254

(22) PCT Filed: Nov. 8, 2022
(86) PCT No.: PCT/JP2022/041539

§ 371 Date: Apr. 3, 2025

(51) Int. Cl. C12M 1/34; C12M 3/00; C12N 5/074

(52) U.S. Cl. CPC C12M 1/343; C12M 3/00; C12N 5/0696; C12N 2506/45

(57) ABSTRACT

Prediction of a differentiation level of a cultured cell is performed non-invasively and simply in an early stage from the end of differentiation induction. A prediction method includes collecting the culture supernatant of the prediction target cultured cell after the start of differentiation induction and before the end of differentiation induction, measuring the content of HICA in the culture supernatant of the collected prediction target cultured cell (step S22), and inputting the content of HICA in the culture supernatant of the prediction target cultured cell measured in the prediction model to predict a differentiation level of the prediction target cultured cell (step S23).

### TECHNICAL FIELD

The present disclosure relates to a prediction method and a prediction device for predicting a differentiation level of a cultured cell.

### BACKGROUND ART

In the production of a cell product, it is important to be able to detect at an early stage an abnormality affecting the quality of the cultured cell being produced in order to reduce energy consumption, waste, and loss cost and to reduce both environmental load and economic load. In order to evaluate the cultured cells at an early stage, it is required to perform the evaluation not at the time of completion of the production of the cell product but in the middle of the production. Furthermore, in the evaluation of the cultured cells during production, a non-invasive method is required in order to prevent contamination in the culture system and partial destruction of the product associated with cell collection.

Examples of the cell product include dopaminergic neural progenitor cells which are obtained by inducing differentiation of induced pluripotent stem cells (iPS cells) and are under clinical trials as cells for treatment of Parkinson's disease. In the production of dopaminergic neural progenitor cells, the culture is once terminated at a stage of about 12 days from the start of differentiation induction, and cells expressing CORIN protein, which is a dopaminergic neural progenitor cell marker, are sorted and advanced to the final step. At this time, the quality of the cultured cells, that is, the level of differentiation into dopaminergic neural progenitor cells can be evaluated by measuring the expression ratio of CORIN protein. However, as described above, a method capable of predicting the differentiation level at an earlier stage than the end of differentiation induction and non-invasively to the cultured cells is important.

As a method for solving the above problems, a method for measuring components in a culture supernatant discarded in the culture process is considered. As a means for predicting the differentiation efficiency from iPS cells to chondrocytes or neural crest cells, PTL 1 discloses a method for constructing a prediction model that is a mathematical formula for predicting the differentiation efficiency of iPS cells to chondrocytes or neural crest cells from quantitative values of a plurality of low molecular weight compounds by quantifying the plurality of low molecular weight compounds contained in a culture supernatant and performing multivariate analysis of the quantified results.

In addition, NPL 1 discloses that maintenance of an undifferentiated state is predicted by the content of a low molecular weight compound kynurenine in culture supernatants of embryonic stem cells and iPS cells, and that expectation of ectodermal differentiation is predicted by the abundance of 2-aminoadipic acid.

As described in PTL 1 and NPL 1, there is a method for performing quality evaluation through measurement of a low molecular weight compound in a culture supernatant. However, PTL 1 and NPL 1 disclose an effective quality evaluation method for a cell type different from dopaminergic neural progenitor cells, and a new method is still necessary for quality evaluation of dopaminergic neural progenitor cells.

In addition, PTL 2 discloses a device and a method for evaluating the quality of a cell by predicting the differentiation level of the cultured cell into a differentiation target exemplified by dopaminergic neural progenitor cells on the basis of the content of a component in a culture supernatant of the cultured cell, particularly a specific protein in an exosome.

### Citation List

### Patent Literature

PTL 1: WO 2021/005729 A
PTL 2: JP 2021-153504 A

### Non-patent Literature

NPL 1: Yamamoto T. et al., Kynurenine signaling through the aryl hydrocarbon receptor maintains the undifferentiated state of human embryonic stem cells, Science Signaling, 25 Jun. 2019, 12(587)

### SUMMARY OF INVENTION

### Technical Problem

In PTL 2 described above, the differentiation level of a cultured cell into a dopaminergic neural progenitor cell is predicted on the basis of the content of a specific protein in the exosome of a high molecular weight compound. However, in the case of an evaluation method based on the content of a low molecular weight compound, it is expected that the quality evaluation will be more convenient. Furthermore, as a method for evaluating cultured cells, it is desirable that there are a plurality of means having different principles, and in addition to the method of PTL 2, a method for predicting the differentiation level of a cultured cell by a low molecular weight compound that is considered to be more easily measurable is required.

Therefore, an object of the present disclosure is to provide a prediction method and a prediction device capable of predicting the differentiation level of a cultured cell non-invasively and simply at an early stage before the end of differentiation induction.

### Solution to Problem

The prediction method according to the present disclosure is a prediction method for predicting a differentiation level of cultured cells, including: collecting a culture supernatant of a prediction target cultured cell after a start of differentiation induction and before an end of differentiation induction; measuring a content of HICA (2-hydroxyisocaproic acid) in the collected culture supernatant; and inputting the measured content of HICA into a prediction model for predicting a differentiation level of the cultured cell from the content of HICA to predict a differentiation level of the prediction target cultured cell.

### Advantageous Effects of Invention

According to the present disclosure, it is possible to predict the differentiation level of a cultured cell non-invasively and simply in an early stage from the end of differentiation induction.

Objects, configurations, and effects of the present disclosure besides the above description will be apparent through the explanation on embodiments for carrying out the invention described below.

### BRIEF DESCRIPTION OF DRAWINGS

FIG. 1 is a flowchart for explaining a method for creating a prediction model according to an embodiment.

FIG. 2 is a flowchart for explaining a method for predicting a differentiation level of a cultured cell of the embodiment.

FIG. 3 is a conceptual diagram for explaining details of a method for creating a prediction model according to the embodiment.

FIG. 4 is a block diagram illustrating a configuration of a prediction device according to an embodiment.

FIG. 5 is a conceptual diagram of a HICA measurement method using an oxidoreductase of the embodiment.

FIG. 6 is a graph illustrating the correlation between the content of HICA in the culture supernatant on Day 4 of differentiation induction and the expression ratio of CORIN protein as an index of the differentiation level on Day 12 of differentiation induction in the differentiation induction step from iPS cells to dopaminergic neural progenitor cells in a first example.

### DESCRIPTION OF EMBODIMENTS

Hereinafter, various embodiments of the present disclosure will be described with reference to the drawings and examples. However, the embodiments are described as merely exemplary to realize the present disclosure, and not limit the technical scope of the present invention. Further, in each of the drawings, the same reference numerals are given to common configurations.

### Prediction Method for Predicting Differentiation Level of Cultured Cell

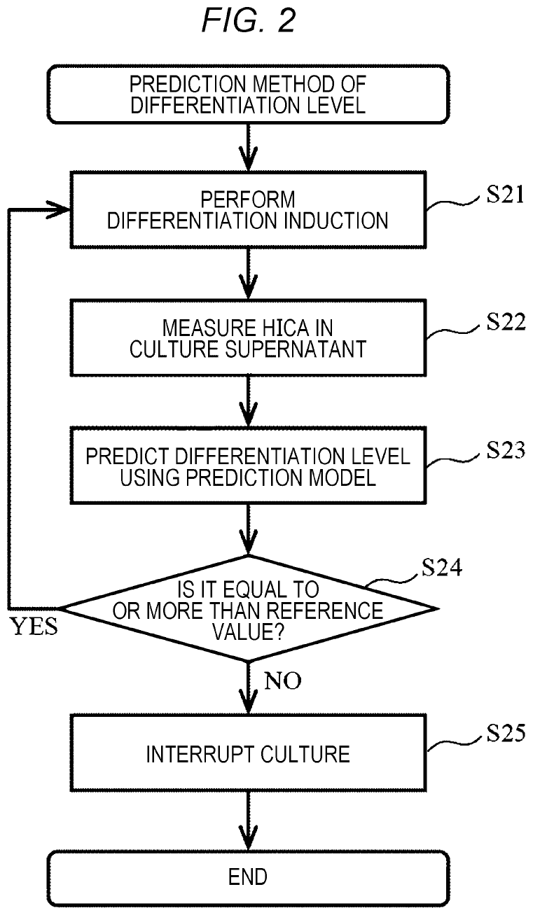

First, a prediction method for predicting the differentiation level of a cell will be described with reference to FIG. 2. The prediction method of the present embodiment is a prediction method for predicting the differentiation level of a cultured cell into a dopaminergic neural progenitor cell, but the differentiation direction of the cultured cell is not limited to dopaminergic neural progenitor cell.

Hereinafter, differentiation of cultured cells into dopaminergic neural progenitor cells will be exemplified, but the direction of differentiation is not particularly limited, and as long as the cells are pluripotent stem cells, neural cells such as nerve cells and glial cells, visceral cells such as liver cells and pancreatic cells, blood cells such as red blood cells and white blood cells, muscle cells such as skeletal muscle and cardiac muscle, immune cells such as T cells, B cells and dendritic cells, epithelial cells, mucosal cells, and stromal cells can be exemplified. In particular, dopaminergic neural progenitor cells and dopaminergic neurons are preferable.

In the prediction method of the present embodiment, the culture supernatant of the cultured cell is collected, the content of HICA, which is a low molecular weight compound, in the culture supernatant is measured, and the differentiation level of the cultured cell is predicted based on the content of HICA measured by the prediction model. Hereinafter, the prediction method for predicting the differentiation level of the cultured cell will be described separately for a creation method for creating a prediction model predicting the differentiation level of the cultured cell based on the content of HICA and a prediction method for predicting the differentiation level of the cultured cell using the prediction model.

In the present disclosure, a compound having a molecular weight of more than 10,000 is defined as a high molecular weight compound, and a compound having a molecular weight of less than 1000 is defined as a low molecular weight compound.

### Creation Method for Creating Prediction Model

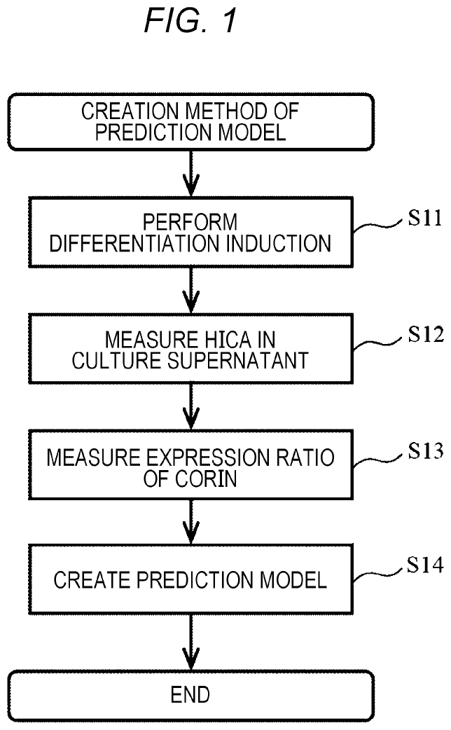

A creation method for creating a prediction model will be described with reference to FIG. 1.

First, differentiation induction of the cultured cells (first cultured cell) (hereinafter, referred to as cultured cells for model creation) prepared for creating the prediction model is performed (step S11).

The culture supernatant discarded as the medium is replaced during differentiation induction is collected, and the content of HICA in the culture supernatant at a specific time point is measured (step S12).

Next, the differentiation level of the cultured cell for model creation at the end of differentiation induction is evaluated. Specifically, the expression ratio of CORIN protein, which is a marker for dopaminergic neural progenitor cells, in the cultured cells for model creation at the end of differentiation induction is measured (step S13). The end of differentiation induction is when a predetermined period has elapsed from the start of differentiation induction, for example, 12 days after the start of differentiation induction. The end of differentiation induction may be 11.5 days, or the end of differentiation induction may be determined based on other criteria.

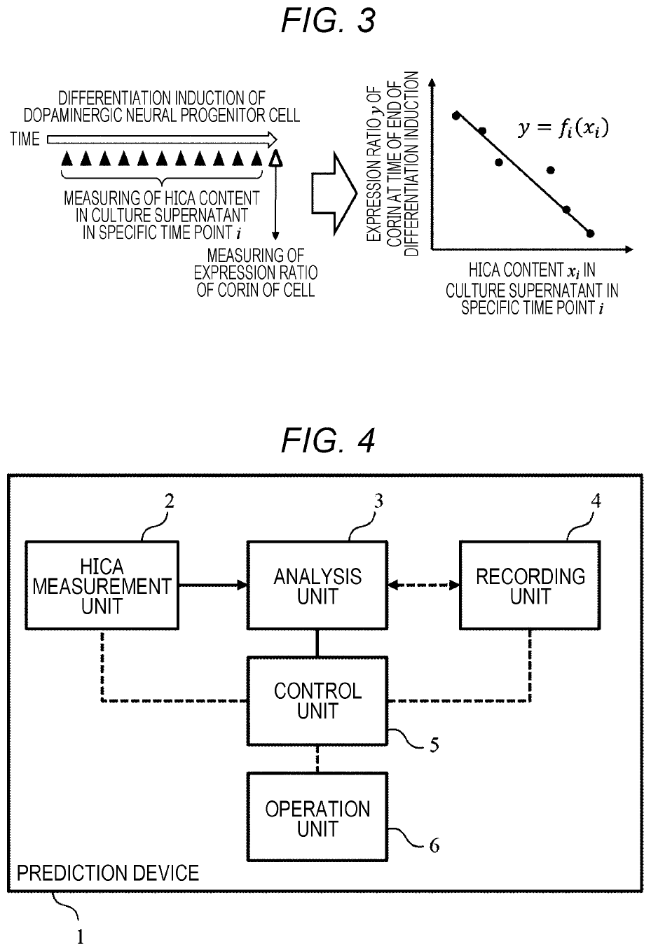

Then, using the content of HICA measured in step S12 and the expression ratio of CORIN protein measured in step S13, a prediction model for predicting the differentiation level of the cultured cell based on the content of HICA is created. A method for creating the prediction model will be described in detail with reference to FIG. 3.

### Prediction Method for Predicting Differentiation Level Using Prediction Model

Next, a prediction method for predicting the differentiation level of the cultured cell using the prediction model will be described with reference to FIG. 2.

First, a prediction target cultured cell (second cultured cell) (hereinafter, referred to as a prediction target cultured cell) prepared, and differentiation induction of the prediction target cultured cell is performed (step S21).

The culture supernatant discarded as the medium is replaced during differentiation induction is collected, and the content of HICA in the culture supernatant at the specific time point is measured (step S22). The specific time point at which HICA is measured in step S12 of FIG. 1 and the specific time point at which HICA is measured in step S22 are preferably set to timings at which the same time has elapsed from the start of differentiation induction, but may be timings at which the same number of days have elapsed from the start of differentiation induction, or may be timings at which the medium is replaced the same number of times after the start of differentiation induction.

Next, the measured content of HICA is input to the prediction model, and the differentiation level of the prediction target cultured cell is predicted (step S23).

In a case where the differentiation level predicted by the prediction model is a predetermined reference value or more, the culture is continued, and in a case where the differentiation level is less than the predetermined reference value, the culture is interrupted (step S24). After the culture is continued, the content of HICA in the culture supernatant at the next specific time point may be input to the prediction model at the next specific time point to predict the differentiation level of the prediction target cultured cell again, and continuation and interruption of the culture may be determined again according to the reference value. In addition, as a result of the determination in the first step S24, in a case where the differentiation level is equal to or greater than the predetermined reference value, the culture may be continued to the end.

The origin of the cultured cells for model creation and the prediction target cultured cells is induced pluripotent stem (iPS) cells.

In the present embodiment, the origin of the cultured cells is human iPS cells, but the kind of the cultured cells is not particularly limited, and examples thereof include pluripotent stem cells such as iPS cells and ES cells, stem cells such as mesenchymal stem cells, and other human-derived cells and animal-derived cells, and the cultured cells may be established or primary cultured cells.

In the present embodiment, the measurement of the low molecular weight compound HICA in the culture supernatant is performed, but the low molecular weight compound may be a component other than HICA as long as it is a low molecular weight compound. For example, in place of HICA, 2-hydroxyvaleric acid, Hypotaurine, β-Alanine, or Ornithine may be used as the low molecular weight compound in the culture supernatant.

As a method for inducing differentiation, a culture protocol in which the medium composition is switched based n a specific regimen as generally disclosed in the literature (Doi, D. et al., Stem cell reports, 2014, 2(3): 337-350) can be used.

In addition, as a method for measuring the content of HICA in the culture supernatant, quantitative analysis using an oxidoreductase, a mass spectrometry method, an immunological method, an aptamer, a charged particle detector, a differential refraction detector, a diode array detector, or an evaporative light scattering detector can be used. Before quantitative analysis, the culture supernatant may be subjected to pretreatment such as solvent extraction, deproteinization, ion exchange, desalination, and concentration. Further, the culture supernatant or the sample obtained by subjecting the culture supernatant to the pretreatment may be subjected to a separation operation by liquid chromatography, gas chromatography, thin layer chromatography, or paper chromatography before the quantitative analysis.

As a method using an oxidoreductase for measuring the content of HICA, electron transfer caused by oxidation or reduction of HICA using an oxidoreductase having HICA as a substrate can be measured by a colorimetric method using an oxidoreductive coloring reagent or an electrode method using an electrode, similarly to a blood glucose level measuring device using an oxidoreductase having glucose as a substrate. As the oxidoreductase having HICA as a substrate, for example, D-2-hydroxyacid dehydrogenase derived from Lactococcus lactis (Chambellon, E. et al., Journal of Bacteriology, 2009, 191(3): 873-881), derived from Ketogulonicigenium vulgare, or derived from Haloferax mediterranei can be used. In addition, an oxidoreductase belonging to the enzyme number 1.1.1.169, 1.1.1.272, or 1.1.1.345 using HICA as a substrate may be used, and furthermore, the oxidoreductase exemplified above or a variant of another oxidoreductase may be used.

### Details of Method for Creating Prediction Model

Next, details of a method for creating a prediction model will be described with reference to FIG. 3.

Culturing is performed a plurality of times in advance, the content xᵢ of HICA in the culture supernatant at a specific time point i after the start of differentiation induction and before the end of differentiation induction and the differentiation level y at the end of differentiation induction are measured, and a prediction model fᵢ(xᵢ) for predicting the differentiation level y from the content of HICA at the specific time point i is created. The specific time point i is set to a timing at which the medium is replaced, and one point is set at the minimum and the number of times the culture medium is replaced is set at the maximum. When performing prediction, the differentiation level can be predicted by measuring the content of HICA at a specific time point i of the cultured cell for which the differentiation level y is desired to be predicted, and inputting the content of HICA at the specific time point i measured in the prediction model fᵢ(xᵢ). Examples of a method for creating a prediction model include, but are not limited to, a method for creating a calibration curve by linear approximation and a method for creating a regression model or a classification model by machine learning.

As a method for predicting the differentiation level y, the expression ratio of a marker of dopaminergic neural progenitor cells in cultured cells at the end of differentiation induction is measured. The marker may be any marker capable of evaluating the differentiation into dopaminergic neural progenitor cells, and for example, CORIN protein (Doi, D. et al., Stem cell reports, 2014, 2(3): 337-350) can be exemplified, but the marker is not limited thereto.

### Prediction Device for Predicting Differentiation Level of Cultured Cell

Next, with reference to FIG. 4, the configuration of a prediction device that measures the content of HICA in the culture supernatant and predicts the differentiation level of the cultured cell using the prediction model described above will be described.

A prediction device 1 includes an HICA measurement unit 2 that measures the content of HICA in the culture supernatant and an analysis unit 3 that predicts the differentiation level of the cultured cell based on the content of HICA. Furthermore, the prediction device 1 may include a recording unit 4 that records data obtained by analysis, a control unit 5 that controls the HICA measurement unit 2, the analysis unit 3, and the recording unit 4, and an operation unit 6 that operates the control unit 5. Furthermore, the prediction device 1 may include the analysis unit 3 without including the HICA measurement unit 2. In this case, the measurement value measured by the external HICA measurement unit 2 is input to the prediction device 1, and the analysis unit 3 of the prediction device 1 predicts the differentiation level of the cultured cell. The analysis unit 3 is a computer system having a processor and a memory, and predicts the differentiation level of the cultured cell using the prediction model described above. Furthermore, the control unit 5 is a computer system capable of communicating with a peripheral device (HICA measurement unit 2, analysis unit 3, recording unit 4, and operation unit 6) of the control unit 5, and includes a processor and a memory. Note that one computer system including a processor and a memory may have the function of the analysis unit 3 and the function of the control unit 5.

The HICA measurement unit 2 has an instrument capable of measuring the content of HICA in the culture supernatant, and may include, for example, a mass spectrometer, a spectrometer, a charged particle detector, a differential refraction detector, a diode array detector, an evaporative scattering detector, or a detector including a reaction cell for HICA measurement using an oxidoreductase and a spectrometer or an electrode that measures electron transfer accompanying an oxidation reaction or a reduction reaction.

### HICA Measurement Method Using Oxidoreductase

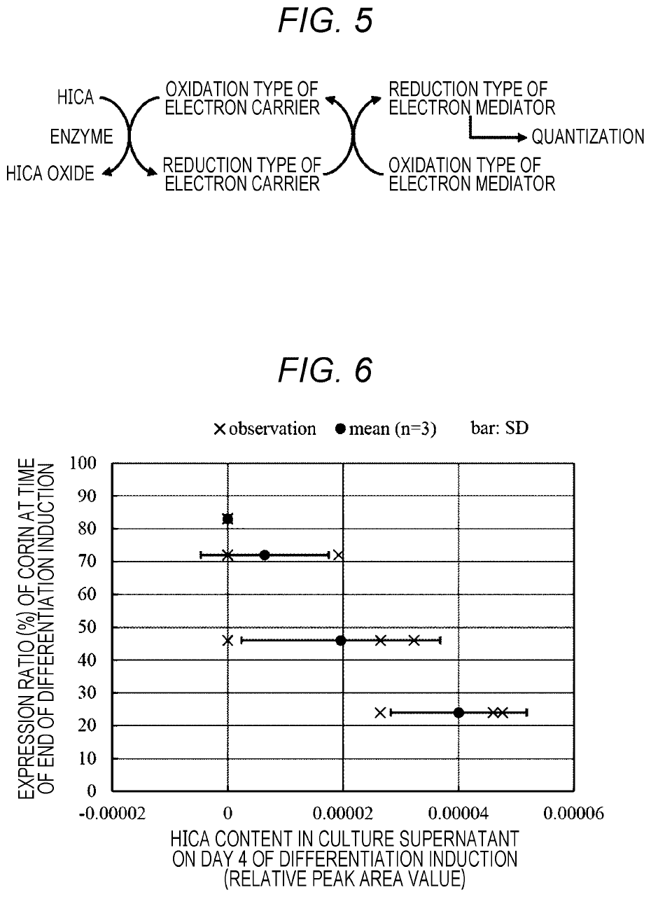

Here, an HICA measurement method using an oxidoreductase will be described with reference to FIG. 5. The HICA measurement unit 2 in the case of using an oxidoreductase includes an electron carrier, exemplified by NAD⁺ or NADP⁺, in addition to an oxidoreductase having HICA as a substrate, or an electron mediator in addition to the electron carrier, and measures electron transfer generated by the oxidation-reduction reaction of HICA. The analysis unit 3 predicts the differentiation level of the cultured cell based on correlation information (prediction model) including the content of HICA received from the HICA measurement unit 2 and the correlation between the content of HICA and the differentiation level recorded in the recording unit 4. The recording unit 4 stores, for example, data obtained from the analysis unit 3 and correlation information (prediction model) including a correlation between the content of HICA and the differentiation level. The recording unit 4 is not particularly limited, but a non-volatile storage device is preferable, and is, for example, a ROM, a flash memory, a magnetic storage device (hard disk drive, floppy disk, magnetic tape, etc.) , or an optical disc. The control of the prediction device 1 may be performed manually or automatically under the control of the control unit 5. An interface for a human to operate the control unit 5 is the operation unit 6, and is, for example, a mouse or a keyboard.

### Effect of This Embodiment

In the embodiment described above, the differentiation level of the cultured cell can be predicted by measuring components in the culture supernatant of the cultured cells after the start of differentiation induction and before the end of differentiation induction. As a result, the differentiation level of the cultured cell can be predicted at an earlier stage than the end of differentiation induction.

In addition, in the embodiment described above, the differentiation level of the cultured cell can be non-invasively predicted by measuring the components in the culture supernatant to be discarded with the replacement of the culture medium.

In addition, in the embodiment described above, since HICA of the low molecular weight compound in the culture supernatant is measured, the differentiation level of the cultured cell can be predicted more easily than the case of handling the high molecular weight compound.

In the embodiment described above, in a case where the differentiation level predicted by the prediction model is less than the predetermined reference value, the culture can be interrupted (step S24), so that the culture can be interrupted at an earlier stage than interrupting the culture at the end of differentiation induction. The ability to detect abnormalities affecting the quality of cultured cells during production at an early stage is effective for reducing energy consumption, waste, and loss costs and reducing both environmental and economic loads.

### First Example

In this example, it is shown that the content of HICA in the culture supernatant on Day 4 of differentiation induction inversely correlates with the expression ratio of CORIN protein, which is one of the indices indicating the differentiation level at the end of differentiation induction, and it is shown that the differentiation level can be predicted by the content of HICA.

In this example, iPS cells have been induced to differentiate into dopaminergic neural progenitor cells, and the content of HICA in the culture supernatant on Day 4 of differentiation induction has been measured. Then, in this example, the expression ratio of CORIN protein has been measured as the differentiation level at the end of differentiation induction, and it has been demonstrated whether the differentiation level at the end of differentiation induction can be predicted from the content of HICA in the culture supernatant on Day 4.

Specifically, differentiation induction has been performed as follows (Doi, D. et al., Stem cell reports, 2014, 2(3): 337-350). 4×10⁵ iPS cell lines 201B7 have been seeded per well in 6-well dishes coated with LM511 E8. When the cells reached confluence, the growth medium (StemFit, Ajinomoto) has been replaced with a differentiation medium (8% KSR, 0.1 mM MEM non-essential amino acids (all ThermoFisher Scientific), sodium pyruvate (Merck), GMEM with 0.1 mM 2-mercaptoethanol (ThermoFisher Scientific)). To promote neural differentiation, LDN193189 (STEMGENT) and A83-01 (Wako) have been added, and further, to induce differentiation of bottom plate cells, purmorphamine and FGF8 (Wako) have been added from Day 1 to Day 7 after medium replacement, and CHIR99021 (Wako/STEMGENT) has been added from Day 3 to Day 12. In this way, differentiation into dopaminergic neural progenitor cells has been induced for 12 days, and the content of HICA in the culture supernatant collected on Day 4 of differentiation induction has been measured.

Specifically, the measurement of the content of HICA has been performed as follows. As analysis pretreatment, 20 μL of an internal standard substance solution (final concentration: 1 mM) has been added to and mixed with 80 μL of culture supernatant, and ultrafiltration has been performed using a filter with a 5 kDa cutoff, and the filtrate has been used as a sample. The sample has been subjected to capillary electrophoresis-time-of-flight mass spectrometry, a peak in which the m/z of the monoisotopic mass peak coincides with the m/z theoretical value of 131.0708 of the monoisotopic ion [M-H]⁻ of HICA (C₆H₁₂O₃) within an error of 10 ppm has been defined as an HICA peak, and the relative peak area value obtained by correcting the peak area value of the HICA peak with the peak area value of the internal standard substance has been defined as the content of HICA.

As a detection method, quantitative analysis using an oxidoreductase, an immunological method, an aptamer, a charged particle detector, a differential refraction detector, a diode array detector, or an evaporative light scattering detector may be used instead of mass spectrometry.

Specifically, the measurement of the expression ratio of CORIN protein at the end of differentiation induction has been performed as follows. Cells on Day 12 of differentiation induction have been detached by TrypLE select (Thermo Fisher Scientific) treatment and pipetting, then stained with an anti-CORIN antibody (R&D Systems) and a secondary antibody (anti-Rat IgG, Abcam), and counted by a cell sorter (PERFLOW, FURUKAWA DENKO).

FIG. 6 is a graph showing the relationship between the content of HICA in the culture supernatant of the cultured cells on Day 4 of differentiation induction and the expression ratio of CORIN protein at the end of differentiation induction. In the example of FIG. 6, for each of the four different cultured cells, the HICA content of three samples of the culture supernatant of the cultured cells on Day 4 of differentiation induction has been measured, and the average thereof has been calculated and plotted. As is clear from the graph of FIG. 6, the lower the content of HICA in the culture supernatant of the cultured cells on Day 4 of differentiation induction, the higher the expression ratio of CORIN protein at the end of differentiation induction.

As described above, it has been found that the content of HICA in the culture supernatant at a specific time point after the start of differentiation induction and before the end of differentiation induction has an inverse correlation with the expression ratio of CORIN protein at the end of differentiation induction. By preparing a calibration curve for this inverse correlation, it has been demonstrated that the CORIN expression ratio at the end of differentiation induction can be predicted in a range of at least 24 to 82% based on the content of HICA in the culture supernatant on Day 4 of differentiation induction.

### Second Example

In the present example, a HICA measurement method using an oxidoreductase having substrate (FIG. 5) performed by the HICA measurement unit 2 of the prediction device 1 illustrated in FIG. 4 will be described.

As the oxidoreductase, D-2-hydroxyacid dehydrogenase (UniProt A2RKB5 or Q9CFY8) derived from Lactococcus lactis has been used. First, the gene encoding the enzyme has been incorporated into an appropriate vector, and expressed using E. coli as a host, and the expressed enzyme has been purified. Selection of an appropriate vector, integration of a gene, expression, and purification can be performed by common technical knowledge of those skilled in the art. The optimum pH of the oxidation-reduction reaction by the enzyme has been 9.0 (Chambellon, E. et al., Journal of Bacteriology, 2009, 191(3): 873-881), and the pH has been adjusted by adding a potassium phosphate buffer having a final concentration of 250 mM to the collected culture supernatant or after pretreating or separating the culture supernatant.

For the oxidation-reduction reaction, the purified enzyme at a final concentration of 4 μg/ml and NAD⁺ at a final concentration of 1 mM have been added to the collected culture supernatant. The temperature of the oxidation-reduction reaction is desirably 55° C., which is a temperature from room temperature to an optimum temperature. As an electron mediator for detecting oxidation of HICA and reduction of NAD⁺ by D-2-hydroxyacid dehydrogenase, 3-(4,5-dimethylthiazol-2-yl)-2,5-diphenyltetrazolium bromide (MTT) at a final concentration of 0.25 mg/mL has been added, the mixture has been allowed to stand for 15 minutes under the pH and temperature conditions, and absorption of formazan (maximum absorption wavelength=565 nm) caused by reduction of MTT by a NAD⁺ reductant (NADH) has been quantified using a general spectrometer, thereby quantifying HICA according to a calibration curve prepared in advance under the same reaction conditions.

The colorimetric method may be performed using an electron mediator such as phosphomolybdic acid or quinones instead of MTT, and the electron transfer caused by the oxidation-reduction reaction may be measured using an electrode. The enzyme is merely an example, and the present invention is not limited to the above. D-2-hydroxyacid dehydrogenase derived from Ketogulonicigenium vulgare or derived from Haloferax mediterranei, or an oxidoreductase having HICA as a substrate and belonging to the enzyme number 1.1.1.169, 1.1.1.272, or 1.1.1.345 may be used. Furthermore, the oxidoreductase exemplified above or a variant of another oxidoreductase may be used. The content of HICA can be quantified using a detector including the oxidoreductase and the reagent described above and quantifying electron transfer associated with oxidation of HICA under the reaction conditions by a colorimetric method or an electrode method. A spectrophotometer can be generally used as a detector used in the colorimetric method, and an electrochemical measurement device can be generally used as a detector used in the electrode method.

In addition, the HICA measurement unit 2 only needs to be able to quantify the content of HICA in the culture supernatant, and a mass spectrometer, a charged particle detector, a differential refraction detector, a diode array detector, or an evaporative light scattering detector may be used instead of the detector, or a colorimetric device using an immunological technique or an aptamer may be used.

The configuration of the prediction device 1 including the HICA measurement unit 2 illustrated in FIG. 4 can include the HICA measurement unit 2, the analysis unit 3 that analyzes the output of the HICA measurement unit 2, and the operation unit 6 that operates the analysis unit 3. For example, in the case of quantitative determination by a colorimetric method, it is possible to include a reaction cell including the oxidoreductase and the reagent, a general spectrophotometer, and the analysis unit 3 that analyzes the output thereof. In this case, for example, the HICA measurement unit 2 includes a reaction cell including the oxidoreductase and the reagent and a spectrophotometer, the control unit 5 that controls the HICA measurement unit 2 and controls the analysis unit 3 and the recording unit 4 is included in the control device, and the operation unit 6 that operates the control unit 5 includes a keyboard or a mouse used for operating the control device.

### Modifications

The present invention is not limited to the above-described embodiments, but various modifications may be contained. For example, the above-described embodiments of the invention have been described in detail in a clearly understandable way, and are not necessarily limited to those having all the described configurations. In addition, some of the configurations of a certain embodiment may be replaced with the configurations of the other embodiments, and the configurations of the other embodiments may be added to the configurations of the subject embodiment. In addition, some of the configurations of each embodiment may be omitted, replaced with other configurations, and added to other configurations.

### Reference Signs List

1 prediction device
2 HICA measurement unit
3 analysis unit
4 recording unit
5 control unit
6 operation unit

1. A prediction method for predicting a differentiation level of a cultured cell, the prediction method comprising:
collecting a culture supernatant of a prediction target cultured cell after a start of differentiation induction and before an end of differentiation induction;
measuring a content of HICA (2-hydroxyisocaproic acid) in the collected culture supernatant; and
inputting the measured content of HICA into a prediction model for predicting a differentiation level of the cultured cell from the content of HICA to predict a differentiation level of the prediction target cultured cell.

2. The prediction method according to claim 1, further comprising:
collecting a culture supernatant of cultured cells for model creation after a start of differentiation induction and before an end of differentiation induction;
measuring a content of HICA in the culture supernatant of the collected cultured cell for the model creation;
evaluating a differentiation level of a cultured cell for the model creation at an end of differentiation induction; and
creating the prediction model using the measured content of HICA in the culture supernatant of the cultured cell for the model creation and the evaluated differentiation level.

3. The prediction method according to claim 2, wherein
the culture supernatant of the cultured cell for the model creation and the culture supernatant of the prediction target cultured cell are collected on Day 4 of differentiation induction.

4. The prediction method according to claim 2, wherein
the cultured cell for model creation and the prediction target cultured cell are derived from human iPS (induced pluripotent stem) cells.

5. The prediction method according to claim 1, wherein
mass spectrometry is used for quantification of a content of HICA in the culture supernatant.

6. The prediction method according to claim 1, wherein
an oxidation-reduction reaction of HICA by an oxidoreductase is used for quantifying a content of HICA in the culture supernatant.

7. The prediction method according to claim 1, wherein
the prediction model is a prediction model that predicts a differentiation level of a cultured cell into a dopaminergic neural progenitor cell, and predicts a differentiation level of the prediction target cultured cell into a dopaminergic neural progenitor cell based on the input content of HICA in the culture supernatant of the prediction target cultured cell.

8. The prediction method according to claim 2, wherein
the evaluating of the differentiation level includes measuring and evaluating an expression ratio of CORIN protein in the cultured cell for the model creation at an end of differentiation induction.

9. A prediction method for predicting a differentiation level of a cultured cell into a dopaminergic neural progenitor cell, the prediction method comprising:
collecting a culture supernatant of a prediction target cultured cell after a start of differentiation induction and before an end of differentiation induction;
measuring a content of a predetermined low molecular weight compound in the collected culture supernatant; and
inputting the measured content of the predetermined low molecular weight compound to a prediction model for predicting the differentiation level of the cultured cell into a dopaminergic neural progenitor cell from the content of the predetermined low molecular weight compound to predict the differentiation level of the prediction target cultured cell into a dopaminergic neural progenitor cell.

10. The prediction method according to claim 9, further comprising:
collecting a culture supernatant of cultured cells for model creation after a start of differentiation induction and before an end of differentiation induction;
measuring a content of the predetermined low molecular weight compound in the culture supernatant of the collected cultured cell for the model creation;
evaluating a differentiation level of a cultured cell for the model creation into a dopaminergic neural progenitor cell at an end of differentiation induction; and
creating the prediction model using the measured content of the predetermined low molecular weight compound in the culture supernatant of the cultured cell for the model creation and the evaluated differentiation level.

11. The prediction method according to claim 10, wherein
the culture supernatant of the cultured cell for the model creation and the culture supernatant of the prediction target cultured cell are collected on Day 4 of differentiation induction.

12. The prediction method according to claim 10, wherein
the cultured cell for model creation and the prediction target cultured cell are derived from human iPS (induced pluripotent stem) cells.

13. The prediction method according to claim 9, wherein
mass spectrometry is used for quantification of a content of the predetermined low molecular weight compound in the culture supernatant.

14. The prediction method according to claim 9, wherein
an oxidation-reduction reaction of the predetermined low molecular weight compound by an oxidoreductase is used for quantifying a content of the predetermined low molecular weight compound in the culture supernatant.

15. The prediction method according to claim 9, wherein
the predetermined low molecular weight compound is HICA (2-hydroxyisocaproic acid).

16. A prediction device for predicting a differentiation level of a cultured cell, the prediction device comprising:
a recording unit that stores a prediction model for predicting a differentiation level of a cultured cell from a content of HICA (2-hydroxyisocaproic acid) in a culture supernatant of the cultured cell after a start of differentiation induction and before an end of differentiation induction; and
an analysis unit that inputs the content of HICA in the culture supernatant of the prediction target cultured cell after a start of differentiation induction and before an end of differentiation induction to the prediction model to predict a differentiation level of the prediction target cultured cell.

17. The prediction device according to claim 16, further comprising:
a measurement unit that measures a content of HICA in a culture supernatant of the prediction target cultured cell after a start of differentiation induction and before an end of differentiation induction, wherein
the analysis unit inputs the content of HICA measured by the measurement unit to the prediction model to predict a differentiation level of the prediction target cultured cell.

18. A prediction device for predicting a differentiation level of a cultured cell into a dopaminergic neural progenitor cell, the prediction device comprising:
a recording unit that stores a prediction model for predicting a differentiation level of a cultured cell into a dopaminergic neural progenitor cell from a content of a predetermined low molecular weight compound in a culture supernatant of the cultured cell after a start of differentiation induction and before an end of differentiation induction; and
an analysis unit that inputs a content of the predetermined low molecular weight compound in a culture supernatant of a prediction target cultured cell after a start of differentiation induction and before an end of differentiation induction to the prediction model to predict a differentiation level of the prediction target cultured cell into a dopaminergic neural progenitor cell.

19. The prediction device according to claim 18, further comprising:
a measurement unit that measures a content of the predetermined low molecular weight compound in a culture supernatant of the prediction target cultured cell after a start of differentiation induction and before an end of differentiation induction, wherein
the analysis unit inputs the content of the predetermined low molecular weight compound measured by the measurement unit to the prediction model to predict a differentiation level of the prediction target cultured cell into a dopaminergic neural progenitor cell.
